(19) United States
(12) Patent Application Publication
(10) Pub. No.: US 2026/0133200 A1
(43) Pub. Date: May 14, 2026

(54) RATIOMETRIC FLUORESCENT PROBE FOR DETECTING A COLORECTAL CANCER MARKER HOMOCYSTEINE WITH HIGH SELECTIVITY AND PREPARATION METHOD THEREFOR

(71) Applicants: Hunan Cancer Hospital, Changsha (CN); THE FOURTH HOSPITAL OF CHANGSHA, Changsha (CN)

(72) Inventors: Huijun ZHOU, Changsha (CN); Dian LIU, Changsha (CN); Ting LI, Changsha (CN); Fei BAI, Changsha (CN); Ying LIU, Changsha (CN); Xiaolin YANG, Changsha (CN); Wu LIU, Changsha (CN); Jianhua WANG, Changsha (CN); Peng YIN, Changsha (CN)

(21) Appl. No.: 19/376,092

(22) Filed: Oct. 31, 2025

(30) Foreign Application Priority Data
Nov. 8, 2024 (CN) 202411588689.4

(51) Int. Cl. G01N 33/574; C07D 491/147; G01N 21/64; G01N 33/533; G01N 33/68

(52) U.S. Cl. CPC G01N 33/57535; C07D 491/147; G01N 21/6428; G01N 33/533; G01N 33/6815; G01N 2021/6439

(57) ABSTRACT

Provided in the present application is a ratiometric fluorescent probe for detecting a colorectal cancer marker homocysteine with high selectivity and a preparation method thereof and relates to the field of analytical chemistry. The ratiometric fluorescent probe has structural formula of:

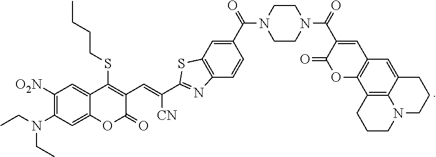 [SMILES: CCCCSc1c(/C=C(\C#N)c2nc3ccc(C(=O)N4CCN(C(=O)c5cc6cc7c8c(c6oc5=O)CCCN8CCC7)CC4)cc3s2)c(=O)oc2cc(N(CC)CC)c([N+](=O)[O-])cc12]

Dual-site binding strategy is adopted in the ratiometric fluorescent probe and the nitro group is introduced into the coumarin fluorophore to achieve specific detection of homocysteine, at the same time, ratiometric detection of homocysteine is achieved by connecting a chromophore having strong green fluorescence through piperazine. Potential interference of background fluorescence can be avoided by the ratio of red fluorescence to green fluorescence, thereby enhancing the sensitivity and accuracy of homocysteine detection. In addition, the probe has the advantages of good selectivity and biocompatibility in detecting homocysteine, and has great application prospects in technical fields such as analytical chemistry, life sciences and biomedicine.

### CROSS-REFERENCE TO RELATED APPLICATIONS

The present application claims priority to Chinese Patent Application No. 202411588689.4, filed on Nov. 8, 2024, the entire disclosure of which is incorporated herein by reference.

### FIELD OF TECHNOLOGY

The present application relates to the field of analytical chemistry, in particular to a ratiometric fluorescent probe for detecting a colorectal cancer marker homocysteine with high selectivity and a preparation method therefor, and a method for detecting homocysteine and imaging homocysteine by using the ratiometric fluorescent probe.

### BACKGROUND

Homocysteine (Hcy) acts as a key intermediate in the circulating process of methionine and the synthetic process of cysteine, and abnormal methylation/transsulfuration metabolism can both lead to an increase of Hcy content, resulting in hyperhomocysteinemia. The serum total Hcy level directly reflects the state of methylation and transsulfuration metabolism of the organism, and overhigh Hcy (>15 μM) can damage cells, tissues and organs and is an independent risk factor or an important risk factor for the occurrence of many chronic diseases (Diabetes and Vascular Disease Research 2007, 4, 143-149), which has become the most accurate independent health index after hypertension, hyperlipidemia and hyperglycemia (International Journal of Molecular Sciences 2016, 17, 1733). Elevated serum total Hcy levels are closely associated with major diseases such as cardiovascular disease, alzheimer's disease, dementia, parkinson's disease, pregnancy syndrome, habitual abortion, osteoporosis and cancer in various epidemiological and clinical relevance analyses (Food Science & Nutrition 2020, 8, 4696-4707; Metabolites, 2021, 11, 37), homocysteine is as an important index of chronic inflammation, and elevated levels thereof promote the occurrence or progression of colorectal cancer through inflammatory mechanisms (Autoimmunity Reviews, 2007, 503-509). In patients with inflammatory bowel disease, elevated homocysteine levels are common and closely associated with colorectal cancer. Meanwhile, serum homocysteine level ia also one of the effective marks of diet-induced inflammation, which is closely related to the recurrence of colorectal adenomas (Cancer Epidemiology, Biomarkers & Prevention, 2010, 19, 1441-1452). It is well known that a ratiometric fluorescent probes is capable of measuring the emission intensity at two different wavelengths, thereby providing a built-in correction for environmental effects (Chemical Society Reviews 2018, 47, 2873-2920; Chemical Society Reviews 2020, 49, 143-179). Compared with the single-emission fluorescent probe, this kind of fluorescent probe eliminates errors caused by various factors, such as the concentration of probe molecules themselves and increases the dynamic range of fluorescence measurement.

Currently, a variety of methods have been developed for the detection of Hcy and viscosity, such as liquid chromatography-mass spectrometry (LCMS), high efficiency liquid chromatography, gas chromatography-mass spectrometry (GCMS), and fluorescence spectroscopy. Among these detection methods, fluorescence probe analysis is generally favored due to its ability of rapid response and high sensitivity, as well as spatial resolution and satisfactory biocompatibility (Angew. Chem. Int. Ed. 2017, 56, 16611-16615; Anal. Chem. 2016, 76, 166-181). Some fluorescent probes for the detection of Hcy have been reported (Angew. Chem. Int. Ed. 2018, 57, 4991; Analyst, 2022, 147, 2470), but ratiometric detection of homocysteine is very difficult. Moreover, homocysteine and cysteine have similar structures and are hard to distinguish. Therefore, it is particularly difficult for high selectivity ratiometric detection without interference from other substances, which is one of the current research challenges.

### SUMMARY

The purpose of the present application is to provide a ratiometric fluorescent probe for detecting a colorectal cancer marker homocysteine with high selectivity and a preparation method therefor, and a method for detecting homocysteine and imaging homocysteine by using the ratiometric fluorescent probe to solve the above problems.

In order to achieve the above purpose, the following technical solutions are adopted in the present application:

A ratiometric fluorescent probe for detecting a colorectal cancer marker homocysteine with high selectivity, and the ratiometric fluorescent probe has a structural formula of:

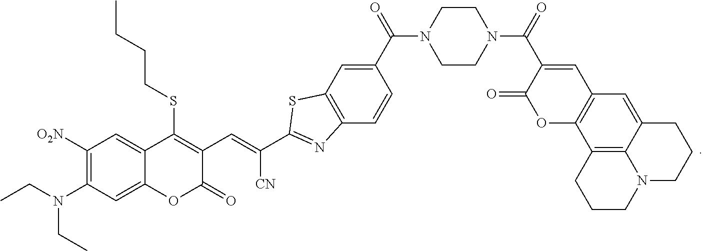 [SMILES: CCCCSc1c(/C=C(\C#N)c2nc3ccc(C(=O)N4CCN(C(=O)c5cc6cc7c8c(c6oc5=O)CCCN8CCC7)CC4)cc3s2)c(=O)oc2cc(N(CC)CC)c([N+](=O)[O-])cc12]

Also provided in the present application is a preparation method for the ratiometric fluorescent probe for detecting a colorectal cancer marker homocysteine with high selectivity, which comprises:
Adding 11-oxo-2,3,6,7-tetrahydro-1H,5H,11H-pyrano[2,3-f]pyrido[3,2,1-ij]quinoline-10-carboxylic acid into anhydrous dichloromethane, then adding 4-dimethylaminopyridine, and adding 1-BOC-piperazine and 1-(3-dimethyl aminopropyl)-3-ethylcarbodiimide hydrochloride after stirring to perform a first reaction, and performing a first separation and purification after the first reaction is completed to obtain tert-butyl 4-(11-oxo-2,3,6,7-tetrahydro-1H, 5H,11H-pyrano[2,3-f]pyrido[3,2,1-ij]quinoline-10-carbonyl) piperazine-1-carboxylate;
adding the tert-butyl 4-(11-oxo-2,3,6,7-tetrahydro-1H, 5H,11H-pyrano[2,3-f]pyrido[3,2,1-ij]quinoline-10-carbonyl) piperazine-1-carboxylate into anhydrous dichloromethane, then adding trifluoroacetic acid to perform a second reaction, removing the solvent after the second reaction is completed, and performing a second separation and purification to obtain 4-(11-oxo-2,3,6,7-tetrahydro-1H, 5H,11H-pyrano[2,3-f]pyrido[3,2,1-ij]quinoline-10-carbonyl) piperazine-1-onium 2,2,2-trifluoroacetate;
adding (E)-2-(2-(4-(butylthio)-7-(diethyl amino)-6-nitro-2-oxo-2H-chromen-3-yl)-1-cyanovinyl)benz o[d]thiazole-6-carboxylic acid to anhydrous dichloromethane, then adding 4-dimethylaminopyridine to perform a third reaction, adding the 4-(11-oxo-2,3,6,7-tetrahydro-1H,5H,11H-pyrano[2,3-f]pyrido[3,2,1-ij]quinoline-10-carbonyl) piperazine-1-onium 2,2,2-trifluoroacetate after the third reaction, and adding 1-(3-dimethylaminopropyl)-3-ethylcarbodiimide hydrochloride to perform a fourth reaction after stirring, removing the solvent after the fourth reaction is completed, and performing a third separation and purification to obtain the ratiometric fluorescent probe.

Preferably, the molar ratio of the (E)-2-(2-(4-(butylthio)-7-(diethylamino)-6-nitro-2-oxo-2H-chromen-3-yl)-1-cyanovinyl)benz o[d]thiazole-6-carboxylic acid to the 4-(11-oxo-2,3,6,7-tetrahydro-1H,5H,11H-pyrano[2,3-f]pyrido[3,2,1-ij]quinoline-10-carbonyl) piperazine-1-onium 2,2,2-trifluoroacetate is 1:1.

Preferably, the first reaction, the second reaction, the third reaction and the fourth reaction are all performed at room temperature.

Preferably, the first separation and purification, the second separation and purification, and the third separation and purification are performed by column chromatography.

Also provided in the present application is a method for detecting homocysteine by using the ratiometric fluorescent probe for detecting a colorectal cancer marker homocysteine with high selectivity, which comprises:

Dissolving the ratiometric fluorescent probe with a solvent and reacting with a system containing homocysteine to measure red fluorescence intensity at 650 nm and green fluorescence intensity at 505 nm, and calculating the ratio of the red fluorescence intensity at 650 nm to the green fluorescence intensity at 505 nm, so as to obtain the content of homocysteine in the system containing homocysteine.

Preferably, the solvent is a mixed solution of dimethyl sulfoxide and PBS.

Preferably, the volume ratio of the dimethyl sulfoxide to the PBS in the mixed solution is 5:5.

Preferably, the measurement is performed at an excitation wavelength of 450 nm.

Also provided in the present application is a method for imaging homocysteine by using the ratiometric fluorescent probe for detecting a colorectal cancer marker homocysteine with high selectivity, which comprises:
Adding the ratiometric fluorescent probe into cells, tissues or living bodies containing homocysteine, and then culturing;
Imaging the homocysteine under the condition of photographing under a confocal fluorescence microscope by a green channel with the wavelength of 490-550 nm and a near-infrared fluorescence channel with the wavelength of 580-700 nm respectively.

Compared with the prior art, the beneficial effects of the present application include:

An ratiometric fluorescent probe for detecting a colorectal cancer marker homocysteine with high selectivity is provided in the present application, and dual-site binding strategy is adopted in the ratiometric fluorescent probe and the nitro group is introduced into the coumarin fluorophore to achieve specific detection of homocysteine, at the same time, ratiometric detection of homocysteine is achieved by connecting a chromophore having strong green fluorescence through piperazine. Two coumarin derivatives are binded together by the probe through piperazine, and coumarin derivative with nitro moiety enables rapid and highly selective detection of homocysteine from various bioactive substances, while strong green fluorescence is emitted by the other coumarin derivative as ratio signal, which can overcome the interference of fluorescence self-quenching and background signals. In addition, the probe has the advantages of rapid real-time response, high sensitivity and good chemical stability. Strong green fluorescence at 505 nm is shown by the probe itself when excited at 430 nm, and when the probe is reacting with homocysteine, not only green fluorescence at 540 nm is shown by the probe but also near-infrared fluorescence at 650 nm is emitted at the same time, and potential interference of background fluorescence can be avoided by the ratio of red fluorescence to green fluorescence, thereby enhancing the sensitivity and accuracy of homocysteine detection. homocysteine can be detected accurately and quantitatively by using the ratio of the fluorescence at 650 nm to the fluorescence at 505 nm, and the detection limit of homocysteine is as low as 28.4 nM. In addition, the probe has the advantages of good selectivity and biocompatibility in detecting homocysteine, and has great application prospects in technical fields such as analytical chemistry, life sciences and biomedicine.

### BRIEF DESCRIPTION OF THE DRAWINGS

To illustrate the technical solutions of the embodiments in the present application more clearly, a brief introduction to the accompanying drawings required in the embodiments will be provided below. It should be understood that the following accompanying drawings only show some embodiments of the present application, and therefore should not be regarded as limiting the scope of the present application.

FIG. 1 is a hydrogen nuclear magnetic resonance spectrum of the ratiometric fluorescent probe provided in the present application;

FIG. 2a and FIG. 2b respectively show an ultraviolet spectrum and a fluorescence spectrum of the ratiometric fluorescent probe in response to homocysteine provided in the present application;

FIG. 3a and FIG. 3b show fluorescence quantitative analysis plots of the ratiometric fluorescent probe in response to homocysteine provided in the present application;

FIG. 4 shows cell graphs of intracellular endogenous homocysteine and the ratiometric fluorescent probe imaged by dual-channel provided in the present application.

### DETAILED DESCRIPTION OF THE EMBODIMENTS

The embodiments of the present application will be described in detail below in conjunction with specific embodiments, but those skilled in the art will understand that the following examples are only used to illustrate the present application, and should not be considered as limiting the scope of the present application. If specific conditions are not indicated in the examples, the conventional conditions or the conditions recommended by the manufacturer shall be followed. If the manufacturers of the reagents or instruments used are not indicated, they are all regular products that are commercially available.

### EXAMPLES

Provided in the example is a ratiometric fluorescent probe for detecting a colorectal cancer marker homocysteine with high selectivity, and the preparation method therefor is as follows:

1. 500.0 mg (1.75 mmol) of 11-oxo-2,3,6,7-tetrahydro-1H,5H,11H-pyrano[2,3-f]pyrido[3,2,1-ij]quinoline-10-carboxylic acid was added into 15 mL of anhydrous dichloromethane, then 21.4 mg of dimethylaminopyridine (DMAP) was added and stirred for 5 min, and subsequently 391.7 mg (2.10 mmol) of 1-BOC-piperazine and 502.2 mg (2.62 mmol) of 1-(3-dimethylaminopropyl)-3-ethylcarbodiimide hydrochloride (EDCI) were added, and the reaction was stirred for overnight at room temperature, and the separation and purification was performed by column chromatography after the reaction was completed to obtain 645.1 mg of tert-butyl 4-(11-oxo-2,3,6,7-tetrahydro-1H,5H,11H-pyrano[2,3-f]pyrido[3,2,1-ij]quinoline-10-carbonyl) piperazine-1-carboxylate with a yield of 81.45%.

2. 500.0 mg (1.1 mmol) of tert-butyl 4-(11-oxo-2,3,6,7-tetrahydro-1H,5H,11H-pyrano[2,3-f]pyrido[3,2,1-ij]quinoline-10-carbonyl) piperazine-1-carboxylate was added into 8 mL of anhydrous dichloromethane, then 2 mL of trifluoroacetic acid was added, and stirred for overnight at room temperature, after the reaction was completed, the reaction system was rotary evaporated to dryness, and the separation and purification was performed by column chromatography to obtain 464.0 mg of 4-(11-oxo-2,3,6,7-tetrahydro-1H,5H,11H-pyrano[2,3-f]pyrido[3,2,1-ij]quinoline-10-carbonyl) piperazine-1-onium 2,2,2-trifluoroacetate with a yield of 90.05%.

3. 100.0 mg (172.81 μmol) of (E)-2-(2-(4-(butylthio)-7-(diethylamino)-6-nitro-2-oxo-2H-chromen-3-yl)-1-cyanovinyl)benz o[d]thiazole-6-carboxylic acid was added to 6 mL of anhydrous dichloromethane, then 10.5 mg of 4-dimethylaminopyridine (DMAP) was added, the reaction was carried out at room temperature for 5 min, and subsequently 80.8 mg (172.81 μmol) of 4-(11-oxo-2,3,6,7-tetrahydro-1H,5H,11H-pyrano[2,3-f]pyrido[3,2,1-ij]quinoline-10-carbonyl) piperazine-1-onium 2,2,2-trifluoroacetate was added, and stirred for 5 min, then 49.7 mg (259.22 μmol) of 1-(3-dimethylaminopropyl)-3-ethylcarbodiimide hydrochloride was added, and stirred for overnight at room temperature, after the reaction was completed, the reaction system was rotary evaporated to dryness, and the separation and purification was performed by column chromatography to obtain 80.0 mg of the ratiometric fluorescent probe of interest with a yield of 50.64%.

The reaction equation is as follows:

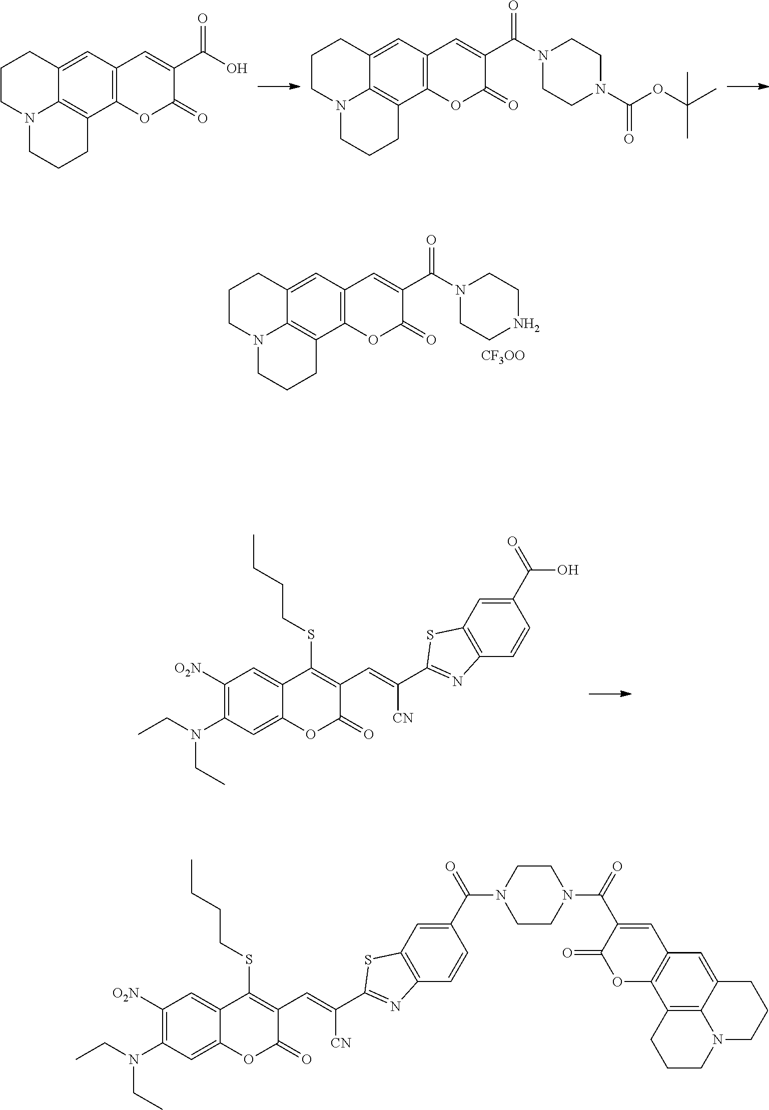

FIG. 1 is a hydrogen nuclear magnetic resonance spectrum of the ratiometric fluorescent probe provided in the present application.

### Experimental Example 1—The Ratiometric Fluorescent Probe for Detection of Homocysteine in an In Vitro Environment

The experimental example is a spectrum property experiment of the ratiometric fluorescent probe, as detailed below:

The bifunctional probe obtained above was dissolved in dimethyl sulfoxide (DMSO) and configured as a probe solution with a concentration of 1 mM and the concentration of homocysteine is configured to 10 mM. The specific test way was as below: 20 μL of 1 mM probe solution was taken, and then 20 μL of 10 mM analyte solution was added, and finally 980 μL of analytically pure DMSO and 980 μL of PBS was added, the volume ratio of organic and aqueous phases was maintained at 5:5 for all tests (total volume 2 mL per sample tested). For example, when the fluorescence intensity of homocysteine at a concentration of 100 μM was required to be tested, the sample was configured as below: 20 μL of 1 mM probe solution and 20 μL of 10 mM homocysteine aqueous solution were taken, 980 μL of analytical pure DMSO and 980 μL of PBS buffer solution were added into a 2 mL sample tube, shaken evenly for 30 minutes at room temperature, then the fluorescence emission intensity can be measured by using the excitation wavelength of 450 nm, and other test operations are similar to the steps above.

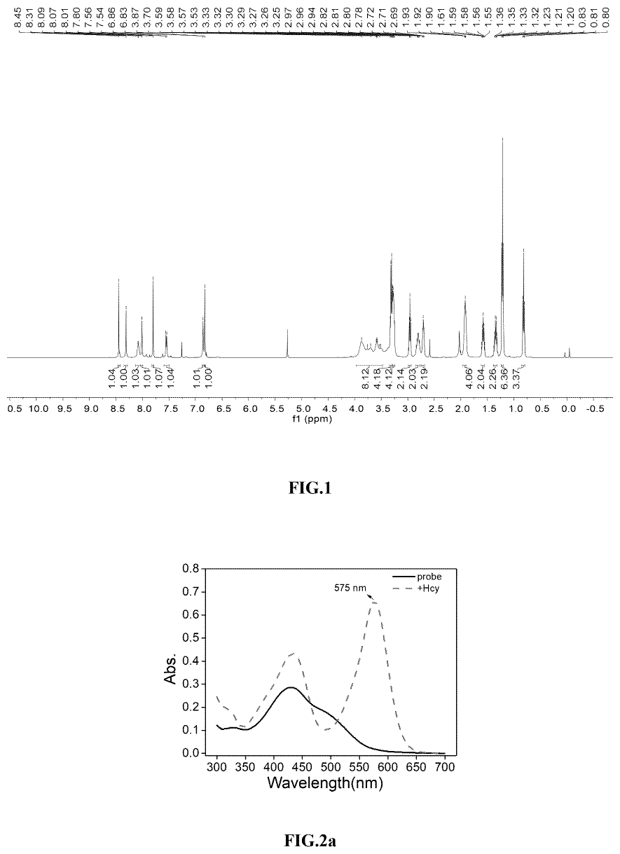

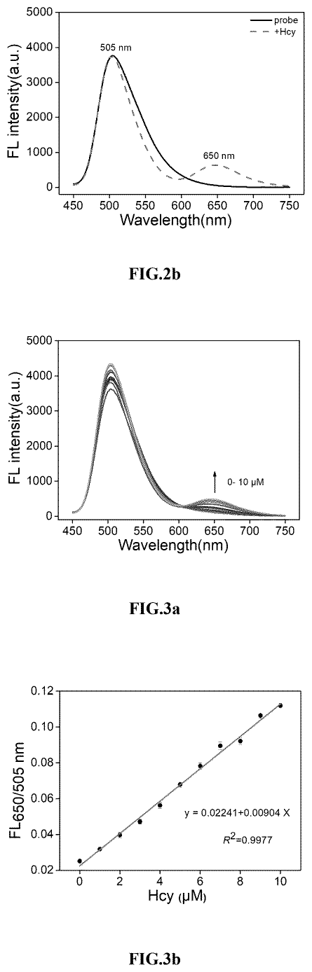

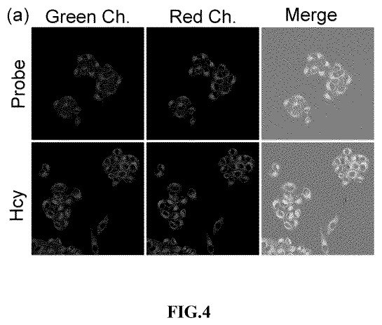

FIG. 2a and FIG. 2b respectively show an ultraviolet spectrum and a fluorescence spectrum of the ratiometric fluorescent probe in response to homocysteine provided in the present application; FIG. 3a and FIG. 3b show fluorescence quantitative analysis plots of the ratiometric fluorescent probe in response to homocysteine provided in the present application.

### Experimental Example 2—Dual-Channel Fluorescence Imaging Analysis of Homocysteine in HepG2 (Hepatoma Cell) Cells

HepG2 cells were subcultured into confocal dish cell culture medium and cultured for 24 hours under standard growth conditions, an appropriate amount of probe (5 μM) was added and continuously cultured for 30 minutes under the standard growth conditions, the homocysteine in the HepG2 cells was imaged under the condition of photographing under a confocal fluorescence microscope by a green channel with the wavelength of 490-550 nm and a near-infrared fluorescence channel with the wavelength of 580-700 nm respectively, the fluorescent probes in the present invention can emit fluorescence with different wavelengths in cells, which indicated that the probes can detect homocysteine in cells by dual-channel ratiometric detection, and the ratiometric fluorescence imaging analysis of homocysteine in cells is successfully achieved.

FIG. 4 shows cell graphs of intracellular endogenous homocysteine and the ratiometric fluorescent probe imaged by dual-channel provided in the present application.

Provided in the present application is a ratiometric fluorescent probe for detecting a colorectal cancer marker homocysteine with high selectivity, and two coumarin derivatives are binded together by the probe through piperazine. One coumarin derivative with nitro moiety can be used to detect homocysteine from various bioactive substances rapidly and selectively, while strong green fluorescence is emitted by the other coumarin derivative as ratio signal. When the probe reacts with homocysteine, green fluorescence at 505 nm and near-infrared fluorescence at 650 nm are emitted under the excitation wavelength of 430 nm, ratiometric detection of homocysteine is carried out by the ratio of the two fluorescence signals. The probe has advantages of good water solubility, fast response speed and large stokes shift and the like, and has great practical application value in fields such as biochemistry and analytical detection.

Finally it should be noted that: the above examples are only used to illustrate the technical solution of the present application, and not to limit the same; although the present application has been described in detail with reference to the aforementioned examples, it will be understood by those skilled in the art that: the technical solutions described in the aforementioned examples may still be modified, or some or all of the technical features may be equivalently substituted; and these modifications or substitutions do not make the essence of the corresponding technical solutions depart from the scope of the technical solutions of the examples of the present application.

What is claimed is:

1. A ratiometric fluorescent probe for detecting a colorectal cancer marker homocysteine with high selectivity, wherein the ratiometric fluorescent probe has a structural formula of:

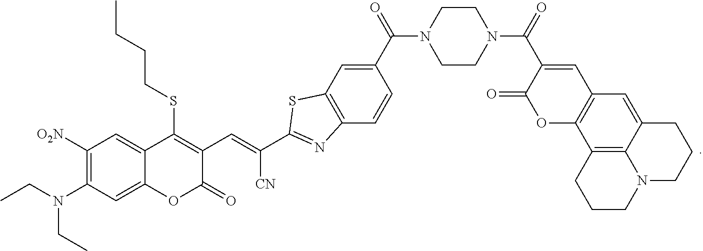 [SMILES: CCCCSc1c(/C=C(\C#N)c2nc3ccc(C(=O)N4CCN(C(=O)c5cc6cc7c8c(c6oc5=O)CCCN8CCC7)CC4)cc3s2)c(=O)oc2cc(N(CC)CC)c([N+](=O)[O-])cc12]

2. A preparation method for the ratiometric fluorescent probe for detecting a colorectal cancer marker homocysteine with high selectivity according to claim 1, which comprises:
adding 11-oxo-2,3,6,7-tetrahydro-1H,5H,11H-pyrano[2,3-f]pyrido[3,2,1-ij]quinoline-10-carboxylic acid into anhydrous dichloromethane, then adding 4-dimethylaminopyridine, and adding 1-BOC-piperazine and 1-(3-dimethylaminopropyl)-3-ethylcarbodiimide hydrochloride after stirring to perform a first reaction, and performing a first separation and purification after the first reaction is completed to obtain tert-butyl 4-(11-oxo-2,3,6,7-tetrahydro-1H,5H,11H-pyrano[2,3-f]pyrido[3,2,1-ij]quinoline-10-carbonyl) piperazine-1-carboxylate;
adding the tert-butyl 4-(11-oxo-2,3,6,7-tetrahydro-1H,5H,11H-pyrano[2,3-f]pyrido[3,2,1-ij]quinoline-10-carbonyl) piperazine-1-carboxylate into anhydrous dichloromethane, then adding trifluoroacetic acid to perform a second reaction, removing the solvent after the second reaction is completed, and performing a second separation and purification to obtain 4-(11-oxo-2,3,6,7-tetrahydro-1H,5H,11H-pyrano[2,3-f]pyrido[3,2,1-ij]quinoline-10-carbonyl) piperazine-1-onium 2,2,2-trifluoroacetate;
adding (E)-2-(2-(4-(butylthio)-7-(diethylamino)-6-nitro-2-oxo-2H-chromen-3-yl)-1-cyanovinyl)benzo[d]thiazole-6-carboxylic acid to anhydrous dichloromethane, then adding 4-dimethylaminopyridine to perform a third reaction, adding the 4-(11-oxo-2,3,6,7-tetrahydro-1H,5H,11H-pyrano[2,3-f]pyrido[3,2,1-ij]quinoline-10-carbonyl) piperazine-1-onium 2,2,2-trifluoroacetate after the third reaction, and adding 1-(3-dimethylaminopropyl)-3-ethylcarbodiimide hydrochloride to perform a fourth reaction after stirring, removing the solvent after the fourth reaction is completed, and performing a third separation and purification to obtain the ratiometric fluorescent probe.

3. The preparation method for the ratiometric fluorescent probe for detecting a colorectal cancer marker homocysteine with high selectivity according to claim 2, wherein the molar ratio of the (E)-2-(2-(4-(butylthio)-7-(diethylamino)-6-nitro-2-oxo-2H-chromen-3-yl)-1-cyanovinyl)benzo[d]thiazole-6-carboxylic acid to the 4-(11-oxo-2,3,6,7-tetrahydro-1H,5H,11H-pyrano[2,3-f]pyrido[3,2,1-ij]quinoline-10-carbonyl) piperazine-1-onium 2,2,2-trifluoroacetate is 1:1.

4. The preparation method for the ratiometric fluorescent probe for detecting a colorectal cancer marker homocysteine with high selectivity according to claim 2, wherein the first reaction, the second reaction, the third reaction and the fourth reaction are all performed at room temperature.

5. The preparation method for the ratiometric fluorescent probe for detecting a colorectal cancer marker homocysteine with high selectivity according to claim 2, wherein the first separation and purification, the second separation and purification, and the third separation and purification are performed by column chromatography.

6. A non-disease diagnosis method for detecting homocysteine by using the ratiometric fluorescent probe for detecting a colorectal cancer marker homocysteine with high selectivity according to claim 1, which comprises:
dissolving the ratiometric fluorescent probe with a solvent and reacting with a system containing homocysteine to measure red fluorescence intensity at 650 nm and green fluorescence intensity at 505 nm, and calculating the ratio of the red fluorescence intensity at 650 nm to the green fluorescence intensity at 505 nm, so as to obtain the content of homocysteine in the system containing homocysteine.

7. The non-disease diagnosis method for detecting homocysteine by using the ratiometric fluorescent probe for detecting a colorectal cancer marker homocysteine with high selectivity according to claim 6, wherein the solvent is a mixed solution of dimethyl sulfoxide and PBS.

8. The non-disease diagnosis method for detecting homocysteine by using the ratiometric fluorescent probe for detecting a colorectal cancer marker homocysteine with high selectivity according to claim 7, wherein the volume ratio of the dimethyl sulfoxide to the PBS in the mixed solution is 5:5.

9. The non-disease diagnosis method for detecting homocysteine by using the ratiometric fluorescent probe for detecting a colorectal cancer marker homocysteine with high selectivity according to claim 8, wherein the measurement is performed at an excitation wavelength of 450 nm.
